(19) United States
(12) Patent Application Publication
(10) Pub. No.: US 2003/0078305 A1
(43) Pub. Date: Apr. 24, 2003

(54) Use of alkoxylated phenol derivatives

(76) Inventors: Helmut-Martin Meier, Ratingen (DE); Hans-Joachim Knackmuss, Leonberg (DE); Paul-Gerhard Rieger, Stuttgart (DE)

Correspondence Address: BAYER CORPORATION, PATENT DEPARTMENT, 100 BAYER ROAD, PITTSBURGH, PA, 15205, US

(21) Appl. No.: 10/223,202

(22) Filed: Aug. 19, 2002

(30) Foreign Application Priority Data
Feb. 25, 2002 (DE) 10207829.7
Aug. 21, 2001 (EP) 01119630.0

(51) Int. Cl. A61K 47/00; A01N 43/72; A01N 25/00

(52) U.S. Cl. 514/772; 504/358

(57) ABSTRACT

Compounds of the general formula (I)

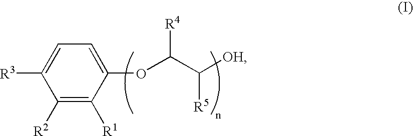

where

R¹, R² and R³ are independently hydrogen, cyclohexyl or phenyl, with the proviso that at least one of R¹ to R³ is not hydrogen,

R⁴ and R⁵ are independently methyl or hydrogen, and

n is from 21 to 40,

which are useful as biodegradable dispersants having good dispersing properties for active pharmaceutical and agrochemical components.

### BACKGROUND OF THE INVENTION

1. Field of the Invention

This invention relates to the use of alkoxylated phenol derivatives as dispersants for active pharmaceutical or agrochemical components, to pharmaceutical or agrochemical formulations including these dispersants, to their preparation and to their use.

2. Brief Description of the Prior Art

Dispersants are needed for many purposes. For example, to disperse dyes which are water-insoluble or sparingly water-soluble compounds, which cannot be used for the dyeing of textiles without the use of a dispersant. The literature describes numerous dispersants which can be used for that purpose. Particularly effective dispersants in this context are ligninsulphonates and naphthalenesulphonates, as documented for example in Chwala/Anger Handbuch der Textilhilfsmittel, 1977, p. 501 ff.

Similarly, alkoxylated phenols and derivatives thereof have already been used for this purpose.

FR-A-2 472 627 explicitly discloses for example ethoxylated phenol derivatives having up to 20 ethylene oxide (EO) units (Examples 1, 2, 3 and 5).

GB-A-20 50 438 explicitly discloses ethoxylated p-phenylphenol having 12 EO units (Example 8).

U.S. Pat. No. 4,328,220 likewise explicitly discloses ethoxylated phenol derivatives having 8 EO units (compound B 30, column 9, line 9).

But all these alkoxylated phenol derivatives have inadequate dispersing effects.

The German patent application bearing the file reference DE 100 42 730.8, unpublished at the priority date of the present invention, discloses alkoxylated phenol derivatives, a process for their preparation and also their use as dispersants in the dyeing of textiles.

Similarly, pharmaceutical and agrochemical formulations frequently require suitable dispersants. Especially in the case of agrochemical formulations, these dispersants should possess good environmental compatibility.

It is an object of the present invention to provide such dispersants for use for pharmaceutical and agrochemical formulations.

### SUMMARY OF THE INVENTION

This object is achieved by the invention by the use of compounds of the general formula (I)

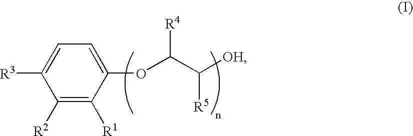

where

R¹, R² and R³ are independently hydrogen, cyclohexyl or phenyl, with the proviso that at least one of R¹ to R³ is not hydrogen,

R⁴ and R⁵ are independently methyl or hydrogen, and

n is from 21 to 40,

as dispersants particularly for active pharmaceutical or agrochemical components.

### DETAILED DESCRIPTION OF THE INVENTION

As set forth above, R¹, R² and R³ in the general formula (I) can independently be hydrogen, cyclohexyl or phenyl, with the proviso that at least one of R¹ to R³ is not hydrogen. Cyclohexyl and phenyl can each also be substituted. Substituents for the cyclohexyl radical include for example C₁-C₄-alkyl, C₁-C₄-alkoxy, halogen, CN or nitro. Preferably, however, the cyclohexyl radical is unsubstituted.

Substituents for the phenyl radical include for example C₁-C₄-alkyl, C₁-C₄-alkoxy, halogen, CN or NO₂. Preferably, however, the phenyl radical is unsubstituted.

n is preferably from 26 to 35 and particularly preferably from 29 to 31.

The preferred phenol radicals are of the formula

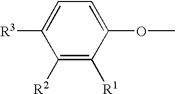

including for example those which are derived from the corresponding phenols of the formula (II) for example those from the group consisting of o-cyclohexylphenol, m-cyclohexylphenol, p-cyclohexylphenol, o-phenylphenol, m-phenylphenol and p-phenylphenol. Particular preference is given to o-phenylphenol and p-phenylphenol, especially p-phenylphenol.

R⁴ and R⁵ are both particularly preferably hydrogen.

Very particular preference is given to the compound of the formula

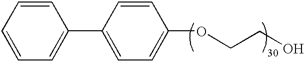

which is a very good dispersant and also readily biodegradable.

The compounds of the formula (I) which are used in the invention can be prepared by reacting phenols of the formula (II)

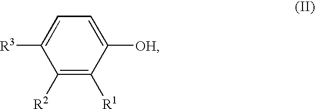

with n mol equivalents of an alkylene oxide of the formula (III)

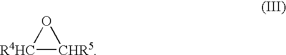

Useful alkylene oxides of the formula (III) preferably include ethylene oxide and/or propylene oxide, especially ethylene oxide.

The alkoxylation is preferably effected in the presence of alkali or alkaline earth metal catalysts, these preferably being used in an amount of 0.1 to 1% by weight, based on the phenol derivative of the formula (II).

Particularly suitable compounds of the formula (II) are o-cyclohexylphenol, m-cyclohexylphenol, p-cyclohexylphenol, o-phenylphenol, m-phenylphenol and p-phenylphenol.

The reaction of the phenol derivatives of the formula (II) with the alkylene oxides of the formula (III), especially ethylene oxide or propylene oxide, is generally carried out by known processes and is described for example in N. Schönfeldt, Grenzflächenaktive Ethylenoxid-Addukte, Stuttgart 1976 which is incorporated herein by reference.

The present invention further provides formulations including

a) at least one active pharmaceutical or agrochemical component,

b) at least one compound of the general formula, and

c) optionally further additives.

The present invention further provides a process for preparing these formulations according to the invention, by mixing

a) at least one active pharmaceutical or agrochemical component

b) with at least one compound of the formula (I) and

c) optionally further additives.

The present invention further provides for the use of the agrochemical formulations according to the invention. Said use includes applying active agrochemical components to plants and/or their habitat.

The formulations according to the invention include at least one active pharmaceutical or agrochemical component.

As used herein, the term “active agrochemical component” comprehends all chemical entities which are customary for the treatment of plants. Specifically they include fungicides, bactericides, insecticides, acaridices, nematicides, herbicides, plant growth regulators, plant nutrients and repellents.

Examples of fungicides which may be mentioned are: 2-aminobutane; 2-anilino-4-methyl-6-cyclopropyl-pyrimidine; 2′,6′-dibromo-2-methyl-4′-trifluoromethoxy-4′-trifluoromethyl-1,3-thiazole-5-carboxanilide; 2,6-dichloro-N-(4-trifluoromethyl benzyl)-benzamide; (E)-2-methoximino-N-methyl-2-(2-phenoxyphenyl)acetamide; 8-hydroxyquinoline sulphate; methyl (E)-2-{2-[6-(2-cyanophenoxy)-pyrimidin-4-yloxy]-phenyl}-3-methoxyacrylate; methyl (E)-methoximino[alpha-(o-tolyloxy)-o-tolyl]-acetate; 2-phenylphenol (OPP), aldimorph, ampropylfos, anilazine, azaconazole, benalaxyl, benodanil, benomyl, binapacryl, biphenyl, bitertanol, blasticidin-S, bromuconazole, bupirimate, buthiobate, calcium polysulphide, captafol, captan, carbendazim, carboxin, quino-methionate, chloroneb, chloropicrin, chlorothalonil, chlozolinate, cufraneb, cymoxanil, cyproconazole, cyprofuram, dichlorophen, diclobutrazol, diclofluanid, diclomezin, dicloran, diethofen-carb, difenoconazole, dimethirimol, dimethomorph, diniconazole, dinocap, diphenylamine, dipyrithion, ditalimfos, dithianon, dodine, drazoxolon, edifenphos, epoxyconazole, ethirimol, etridiazole, fenarimol, fenbuconazole, fenfuram, fenitropan, fenpiclonil, fenpropidin, fenpropimorph, fentin acetate, fentin hydroxide, ferbam, ferimzone, fluazinam, fludioxonil, fluoromide, fluquinconazole, flusilazole, flusulf-amide, flutolanil, flutriafol, folpet, fosetyl-aluminium, fthalide, fuberidazole, furalaxyl, furmecyclox, guazatine, hexachlorobenzene, hexaconazole, hymexazol, imazalil, imibenconazole, iminoctadine, iprobenfos (IBP), iprodione, isoprothiolane, kasugamycin, mancozeb, maneb, mepanipyrim, mepronil, metalaxyl, metconazole, methasulfocarb, methfuroxam, metiram, metsulfovax, myclobutanil, nickel dimethyidithiocarbamate, nitrothal-isopropyl, nuarimol, ofurace, oxadixyl, oxamocarb, oxycarboxin, pefurazoate, penconazole, pencycuron, phosdiphen, pimaricin, piperalin, polyoxin, probenazole, prochloraz, procymidone, propamocarb, propicon-azole, propineb, pyrazophos, pyrifenox, pyrimethanil, pyroquilon, quintozene (PCNB), tebuconazole, tecloftalam, tecnazene, tetraconazole, thiabendazole, thicyofen, thiophanate-methyl, thiram, tolclophos-methyl, tolylfluanid, triadimefon, triadimenol, triazoxide, trichlamide, tricyclazole, tridemorph, triflumizole, triforine, triticonazole, validamycin A, vinclozolin, zineb, ziram, 8-tert-butyl-2-(N-ethyl-N-n-propyl-amino)-methyl-1,4-dioxa-spiro-[4,5]decane, N-(R)-[1-(4-chlorophenyl)-ethyl]-2,2-dichloro-1-ethyl-3t-methyl-1r-cyclopropanecarboxamide (diastereomer mixture or individual isomers), 1-methylethyl [2-methyl-1-[[[1-(4-methylphenyl)-ethyl]-amino]-carbonyl]-propyl]-carbamate, N-(2,3-dichloro-4-hydroxy)-1-methyl-cyclohexyl-1-carboxanilide, 2-[2-(1-chloro-cyclopropyl)-3-(2-chlorophenyl)-2-hydroxypropyl]-2,4-dihydro-[1,2,4]-triazole-3-thione, 1-(3,5-dimethyl-isoxazole-4-sulphonyl)-2-chloro-6,6-difluoro-[1,3]-dioxolo-[4,5-f]-benzimidazole and (5,6-dihydro-1,4,2-dioxazin-3-yl)-{2-[[6-(2-chloro-phenoxy)-5-fluoro-4-pyrimidinyl]-oxy]phenyl}-methanone O-methyloxime.

Examples of bactericides which may be mentioned are: bronopol, dichlorophen, nitrapyrin, nickel dimethyidithiocarbamate, kasugamycin, octhilinone, furancarboxylic acid, oxytetracyclin, probenazole, streptomycin, tecloftalam, copper sulphate and other copper preparations.

Examples of insecticides, acaricides and nematicides which may be mentioned are: abamectin, acephate, acrinathrin, alanycarb, aldicarb, alphamethrin, amitraz, avermectin, AZ 60541, azadirachtin, azinphos A, azinphos M, azocyclotin, Bacillus thuringiensis, 4-bromo-2-(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, BPMC, brofenprox, bromophos A, bufencarb, buprofezin, butocarboxin, butylpyridaben, cadusafos, carbaryl, carbofuran, carbophenothion, carbosulfan, cartap, chloethocarb, chloroethoxyfos, chlorofenvinphos, chlorofluazuron, chloro- mephos, N-[(6-chloro-3-pyridinyl )-methyl]-N′-cyano-N-methyl-ethan-imidamide, chloropyrifos, chloropyrifos M, cis-resmethrin, clocythrin, clofentezine, cyanophos, cycloprothrin, cyfluthrin, cyhalothrin, cyhexatin, cypermethrin, cyromazine, deltamethrin, demeton-M, demeton-S, demeton-S-methyl, diafenthiuron, diazinon, dichlofenthion, dichlorvos, dicliphos, dicrotophos, diethion, diflubenzuron, dimethoate, dimethylvinphos, dioxathion, disulfoton, edifenphos, emamectin, esfenvalerate, ethiofencarb, ethion, ethofenprox, ethoprophos, etrimphos, fenamiphos, fenazaquin, fenbutatin oxide, fenitrothion, fenobucarb, fenothiocarb, fenoxycarb, fenpropathrin, fenpyrad, fenpyroximate, fenthion, fenvalerate, fipronil, fluazinam, fluazuron, flucycloxuron, flucythrinate, flufenoxuron, flufenprox, fluvalinate, fonophos, formothion, fosthiazate, fubfenprox, furathiocarb, HCH, heptenophos, hexaflumuron, hexythiazox, imidacloprid, iprobenfos, isazophos, isofenphos, isoprocarb, isoxathion, ivermectin, lambda-cyhalothrin, lufenuron, malathion, mecarbam, mevinphos, mesulfenphos, metaldehyde, methacrifos, methamidophos, methidathion, methiocarb, methomyl, metolcarb, milbemectin, monocrotophos, moxidectin, naled, NC 184, nitenpyram, omethoate, oxamyl, oxydemethon M, oxydeprofos, parathion A, parathion M, permethrin, phenthoate, phorate, phosalone, phosmet, phosphamidon, phoxim, pirimicarb, pirimiphos M, pirimiphos A, profenofos, promecarb, propaphos, propoxur, prothiofos, prothoate, pymetrozin, pyrachlophos, pyridaphenthion, pyresmethrin, pyrethrum, pyridaben, pyrimidifen, pyriproxifen, quinalphos, salithion, sebufos, silafluofen, sulfotep, sulprofos, tebufenozide, tebufenpyrad, tebupirimiphos, teflubenzuron, tefluthrin, temephos, terbam, terbufos, tetrachlorvinphos, thiafenox, thiomethoxam, thiodicarb, thiofanox, thiomethon, thionazin, thuringiensin, tralomethrin, transfluthrin, triarathen, triazophos, triazuron, trichlorfon, triflumuron, trimethacarb, vamidothion, XMC, xylylcarb, zetamethrin.

Examples of herbicides which may be mentioned are: anilides such as, for example, diflufenican and propanil; arylcarboxylic acids such as, for example, dichloropicolinic acid, dicamba and picloram; aryloxyalkanoic acids such as, for example, 2,4-D, 2,4-DB, 2,4-DP, fluroxypyr, MCPA, MCPP and triclopyr; aryloxy-phenoxy-alkanoic esters such as, for example, diclofop-methyl, fenoxaprop-ethyl, fluazifop-butyl, haloxyfop-methyl and quizalofop-ethyl; azinones such as, for example, chloridazon and norflurazon; carbamates such as, for example, chlorpropham, desmedipham, phenmedipham and propham; chloroacetanilides such as, for example, alachlor, acetochlor, butachlor, metazachlor, metolachlor, pretilachlor and propachlor; dinitroanilines such as, for example, oryzalin, pendimethalin and trifluralin; diphenyl ethers such as, for example, acifluorfen, bifenox, fluoroglycofen, fomesafen, halosafen, lactofen and oxyfluorfen; ureas such as, for example, chlortoluron, diuron, fluometuron, isoproturon, linuron and methabenzthiazuron; hydroxylamines such as, for example, alloxydim, clethodim, cycloxydim, sethoxydim and tralkoxydim; imidazolinones such as, for example, imazethapyr, imazamethabenz, imazapyr and imazaquin; nitriles such as, for example, bromoxynil, dichlobenil and ioxynil; oxyacetamides such as, for example, mefenacet; sulphonylureas such as, for example, amidosulfuron, bensulfuron-methyl, chlorimuron-ethyl, chlorsulfuron, cinosulfuron, metsulfuron-methyl, nicosulfuron, primisulfuron, pyrazosulfuron-ethyl, thifensulfuron-methyl, triasulfuron and tribenuron-methyl; thiolcarbamates such as, for example, butylates, cycloates, diallates, EPTC, esprocarb, molinates, prosulfocarb, thiobencarb and triallates; triazines such as, for example, atrazine, cyanazine, simazine, simetryne, terbutryne and terbutylazine; triazinones such as, for example, hexazinone, metamitron and metribuzin; others such as, for example, aminotriazole, 4-amino-N-(1,1-dimethylethyl)-4,5-dihydro-3-(1-methylethyl)-5-oxo-1H-1,2,4-triazole-1-carboxamide, benfuresate, bentazone, cinmethylin, clomazone, clopyralid, difenzoquat, dithiopyr, ethofumesate, fluorochloridone, glufosinate, glyphosate, isoxaben, pyridate, quinchlorac, quinmerac, sulphosate and tridiphane.

Plant growth regulators which may be mentioned are chlorocholine chloride and ethephon.

Examples of plant nutrients which may be mentioned are customary inorganic or organic fertilizers for providing plants with macro- and/or micronutrients.

Examples of repellents which may be mentioned are diethyltolylamide, ethylhexanediol and butopyronoxyl.

Particularly preferred examples of fungicides which may be mentioned are the active compounds of the formulae

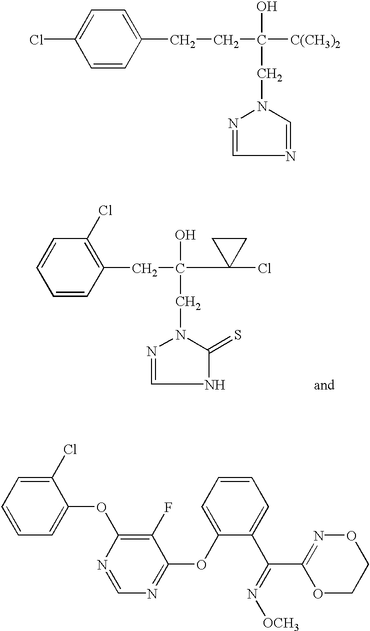

The formulations according to the invention further include at least one of the compounds of the general formula (I).

Suitable additives which may be present in the formulations according to the invention which are different from the compounds of the general formula (I) are all customary formulation auxiliaries such as, for example, organic solvents, emulsifiers, dispersants, preservatives, colorants, fillers and also water.

Suitable organic solvents are all customary organic solvents which dissolve the agrochemically active compounds used well. These are preferably aliphatic and aromatic, optionally halogenated hydrocarbons, such as toluene, xylene, Solvesso®, mineral oils, such as white spirit, petroleum, alkylbenzenes and spindle oil, furthermore carbon tetrachloride, chloroform, methylene chloride and dichloromethane, moreover esters, such as ethyl acetate, furthermore lactones, such as butyrolactone, moreover lactams, such as N-methylpyrrolidone, N-octyl-pyrrolidone and N-methylcaprolactam, and also alkanecarboxamides, such as N,N-dimethyl-decanecarboxamide and N,N-dimethyl-octanecarboxamide, and also dimethylformamide.

Suitable emulsifiers are customary surfactants which are present in formulations of active agrochemical components and are different from compounds of the formula (I). Examples which may be mentioned are ethoxylated nonylphenols, polyethylene glycol ethers of linear alcohols, reaction products of alkylphenols with ethylene oxide and/or propylene oxide, furthermore fatty esters, alkylsulphonates, alkyl sulphates, aryl sulphates, ethoxylated arylalkylphenols, such as, for example, tristyryl-phenol ethoxylate having on average 16 ethylene oxide units per molecule, furthermore ethoxylated and propoxylated arylalkylphenols and sulphated or phosphated arylalkylphenol ethoxylates or ethoxy- and propoxylates.

Suitable dispersants are all substances which are customarily used for this purpose in crop protection compositions and are different from compounds of the formula (I). These are preferably natural and synthetic water-soluble polymers, such as gelatin, starch and cellulose derivatives, in particular cellulose esters and cellulose ethers, further polyvinyl alcohol, polyvinylpyrrolidone, polyacrylic acid, polymethacrylic acid and copolymers of (meth)acrylic acid and (meth)acrylic esters, and furthermore also copolymers of methacrylic acid and methacrylic ester which are neutralized with alkali metal hydroxide.

Suitable preservatives are all substances which are customarily present for this purpose in crop treatment compositions. Examples which may be mentioned are Preventol® and Proxel®.

Suitable colorants are all inorganic or organic colorants which are customarily used for preparing crop protection compositions. Examples which may be mentioned are titanium dioxide, colour black, zinc oxide and blue pigments.

Suitable fillers are all substances which are customarily used for this purpose in crop protection compositions. These are preferably inorganic particles, such as carbonates, silicates and oxides having an average particle size of from 0.005 to 5 μm, particularly preferably from 0.02 to 2 μm. Examples which may be mentioned are silicon dioxide, finely divided silicic acid, silica gels, and natural and synthetic silicates and alumosilicates.

The content of the individual components in the formulations according to the invention can be varied within a relatively wide range. Thus, the concentrations

of active agrochemical components are generally between 1 and 90% by weight, preferably between 5 and 30% by weight

of compounds of the general formula (I) are generally between 1 and 90% by weight, preferably between 10 and 50% by weight and

of additives are generally between 0 and 98% by weight, preferably between 20 and 85% by weight.

The formulations according to the invention are prepared by mixing the components in the particular ratios desired. If the active agrochemical component is a solid, it is generally employed in finely ground form or in the form of a solution or suspension in an organic solvent. If the active agrochemical component is liquid, it is frequently not necessary to use an organic solvent. It is furthermore possible to employ a solid active agrochemical component in the form of a melt.

When carrying out the process according to the invention, the temperatures can be varied within a certain range. In general, the process is carried out at temperatures between 0° C. and 80° C., preferably between 10° C. and 60° C.

The process according to the invention is generally carried out by mixing the compound of the general formula (I) with one or more active components and, if appropriate, with additives, by stirring intensively. The components can be mixed with one another in any order. In a preferred variant of the process according to the invention, however, the compound of general formula (I) is mixed with one or more active components and with other additives, and the resulting premix is dispersed in water, giving emulsions, suspensions or solutions.

Suitable for carrying out the process according to the invention is customary apparatus which are employed for preparing such formulations.

The formulations according to the invention can in the case of actin agrochemical ingredients be applied in the forms of preparation which are customary for liquid preparations, either as such or after prior dilution with water, i.e., for example, as emulsions, suspensions or solutions. The application is carried out by customary methods, i.e., for example, by spraying, watering or injecting.

The application rate of the agrochemical formulations according to the invention can be varied within a relatively wide range. It depends on the respective active agrochemical components and their content in the formulations.

Using the formulations according to the invention, it is possible to apply active agrochemical components in a particularly advantageous manner to plants and/or their habitat. Good dispersion of the actives components is achieved in that the tendency of solid actives to crystallize is reduced. Moreover, the dispersants according to the invention exhibit ready biodegradability, which is an important criterion for environmental compatibility. This applies especially to agrochemical formulations, since in use the products pass into the soil and should not accumulate in the soil.

### EXAMPLES

### Preparation Example 1

170.2 g of p-phenylphenol (1 mol) are dewatered at 170° C. in an autoclave in the course of 10 minutes under a high vacuum and then reacted at 2.5 bar/170° C. with 1320 g of ethylene oxide (30 mol) in the presence of 1.7 g of potassium hydroxide over 10 hours. The end product is used in the form of an aqueous solution as a dispersant without further work-up. The properties with regard to biodegradability and dispersing power are shown in the table which follows.

### Preparation Examples 2 to 4

Further compounds of the general formula (I) are prepared similarly to Example 1, except that the reaction temperature is varied from 120° C. to 170° C. and the pressure from 2.0 to 4 bar depending on the starting material. The properties with regard to biodegradability and dispersing power are shown in the table which follows. The dispersant test employed is also informative about the dispersing power of the compounds of the formula (I) with regard to agrochemical or pharmaceutical substances.

### Comparative Examples 1 and 2

Ethoxylated p-phenylphenol (Example 3 of FR-A 2 472 627; 20 mol equivalent of EO) and o-phenylphenol (Example 2 of FR-A 2 472 627; 15 mol equivalent of EO) are prepared and compared with the compounds of Examples 1 and 2 respectively. Surprisingly, the compounds according to the invention had a distinctly higher DF value.

The dispersing properties were determined by means of a hereinbelow described dispersant test.

The biodegradability was measured using a test according to OECD 301 A ISO 7827 (DIN 7827) (measurement of the decrease in organically bound carbon).

Under these guidelines, an organic substance is classified as “readily biodegradable” in any OECD 301 A to F test when the degradation amounts to >60%.

Dispersant Test

5.0 g of Resolin Blue BBLS (C.I. Disperse Blue 56) and 10.0 g of sodium acetate (trihydrate) were bulked with demineralized water to 5 000 ml. During stirring, the pH was adjusted to 4.5 with acetic acid.

100 ml lots of this dye liquor were used together with 1 g/l of the dispersant to be tested. The resultant liquor was subsequently subjected to a 120° C. dyeing program on a Spectra Dye machine (pot dyeing). The liquor was then filtered through a 5.5 cm glass slotted sieve suction filter (with 3 S&S BF filter discs).

A 1 ml sample was taken from both the liquor prior to filtration and from the filtrate and dissolved in 20 ml of acetone. The dye quantities contained in the acetonic solutions were evaluated in UV-VIS (1 cm cell, 610 nm). The relevant quantity is considered to be the dispersion factor (DF)=absorbance of filtrate/absorbance of made-up liquor. A DF of 1 indicates a very good dispersant, while a DF of 0 indicates a dispersant lacking utility.

TABLE

| | | Reaction product | Biodegrad- | |
|---|---|---|---|---|
| | | with n mol | ability | |
| | Phenol derivative of | equivalent of | to OECD | |
| Example | formula (II) | ethylene oxide | 301 A | DF |
| Inventive 1 | p-Phenylphenol | 30 | 94% | 1.0 |
| Comparative | p-Phenylphenol | 20 | 98% | 0.3 |
| 1 | | | | |
| Inventive 2 | o-Phenylphenol | 26 | 67% | 0.9 |
| Comparative | o-Phenylphenol | 15 | 67% | 0.5 |
| 2 | | | | |
| Inventive 3 | o-Cyclohexylphenol | 21 | 74% | 0.9 |
| Inventive 4 | p-Cyclohexylphenol | 21 | 68% | 0.9 |

### Use Example

A crop protection formulation according to the invention has the following composition:

2.45 g of tebuconazole,

3.50 g of the dispersant of Example 1, and

10 3.55 g of N-methylcaprolactam.

A finely divided emulsion forms in water and can be used as a spray liquor.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for preparing formulations of active pharmaceutical or agrochemical components comprising dispersing the active components with compounds of the formula (I) 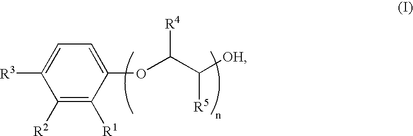
where
R¹, R² and R³ are independently hydrogen, cyclohexyl or phenyl, with the proviso that at least one of R¹ to R³ is not hydrogen,
R⁴ and R⁵ are independently methyl or hydrogen, and
n is from 21 to 40.

2. The process according to claim 1, wherein n is from 26 to 35 in the formula (I).

3. The process according to claim 1, wherein R³ is phenyl in the formula (I).

4. The process according to claim 1, wherein in the formula (I), at least one of R¹ to R³ is cyclohexyl and n is from 21 to 40.

5. Formulations comprising
a) at least one active pharmaceutical or agrochemical component,
b) at least one compound of the general formula (I), and
c) optionally further additives.

6. Formulation according to claim 5, wherein the compound of the formula (I) comprises 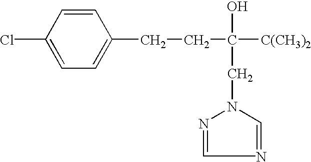
as an active agrochemical component.

7. Formulations according to claim 5, wherein the compound of the formula (I) comprises 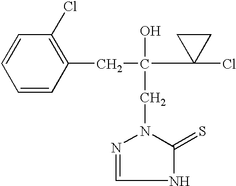
as an active agrochemical component.

8. Formulations according to claim 5, wherein the compound of the formula (I) comprises 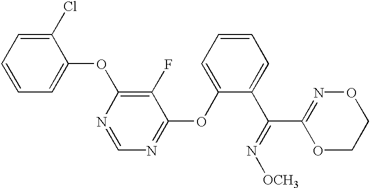
as an active agrochemical component.

9. Process for preparing formulations according to claim 5, comprising mixing
a) at least one active pharmaceutical or agrochemical component
b) with at least one compound of the formula (I) and
c) optionally further additives.

10. A process for using agrochemical formulations according to claim 5 comprising applying the formulations to plants and/or their habitat.
